(19) United States
(12) Patent Application Publication
(10) Pub. No.: US 2024/0208976 A1
(43) Pub. Date: Jun. 27, 2024

(54) PROCESS FOR THE PREPARATION OF 7-(4-CHLOROBENZYL)-1-(3-HYDROXYPROPYL)-3-METHYL-8-(3-(TRIFLUOROMETHOXY)-PHENOXY)-3,7-DIHYDRO-1H-PURINE-2,6-DIONE

(71) Applicants: Boehringer Ingelheim International GnmH, Ingelheim am Rhein (DE); Hydra Biosciences, LLC, Belmont, MA (US)

(72) Inventors: Martin Butschies, Ingelheim am Rhein (DE); Juergen Daeubler, Ingelheim am Rhein (DE); Julia Feile, Ingelheim am Rhein (DE); Annika Gille, Ingelheim am Rhein (DE); Jannik Hahn, Ingelheim am Rhein (DE); Alexander Haydl, Ingelheim am Rhein (DE); Guenther Huchler, Ingelheim am Rhein (DE); Christoph Kressierer, Ingelheim am Rhein (DE); Josef Mesaki Landu, Ingelheim am Rhein (DE); Sue Lauterbach, Ingelheim am Rhein (DE); Zeno Andreas Leuter, Ingelheim am Rhein (DE); Huayu Li, Ridgefield, CT (US); Jon Charles Lorenz, Ridgefield, CT (US); Markus Ostermeier, Ingelheim am Rhein (DE); Michael Pangerl, Ingelheim am Rhein (DE); Hartmut Schmidt, Ingelheim am Rhein (DE); Sascha Wagner, Ingelheim am Rhein (DE); Uwe Wallmen, Ingelheim am Rhein (DE); Michael Wedel, Ingelheim am Rhein (DE); Jiang-Ping Wu, Danbury, CT (US); Ewa Zimny, Ridgefield, CT (US)

(73) Assignees: Boehringer Ingelheim International GmbH, Ingelheim am Rhein (DE); Hydra Biosciences, LLC, Belmont, MA (US)

(21) Appl. No.: 18/390,926

(22) Filed: Dec. 20, 2023

(30) Foreign Application Priority Data
Dec. 20, 2022 (EP) EP 22 214 917.1

(51) Int. Cl. C07D 473/04

(52) U.S. Cl. CPC C07D 473/04

(57) ABSTRACT

The present invention relates to a novel process for the preparation of 7-(4-chlorobenzyl)-1-(3-hydroxypropyl)-3-methyl-8-(3-(trifluoromethoxy)phenoxy)-3,7-dihydro-1H-purine-2,6-dione (compound (I)):

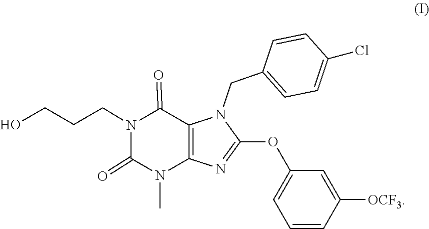 [SMILES: Cn1c(=O)n(CCCO)c(=O)c2c1nc(Oc1cccc(OC(F)(F)F)c1)n2Cc1ccc(Cl)cc1]

### FIELD OF THE INVENTION

The present invention relates to a novel process for the preparation of 7-(4-chlorobenzyl)-1-(3-hydroxypropyl)-3-methyl-8-(3-(trifluoromethoxy)-phenoxy)-3,7-dihydro-1H-purine-2,6-dione (compound (I)):

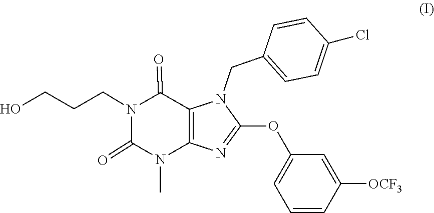 [SMILES: Cn1c(=O)n(CCCO)c(=O)c2c1nc(Oc1cccc(OC(F)(F)F)c1)n2Cc1ccc(Cl)cc1]

### BACKGROUND OF THE INVENTION

Compound I is a potent and selective inhibitor of the transient receptor potential (TRP) cation channel subfamily C, member 5 (TRPC5) and was first described in WO®2014/143799 (Example 7a, p. 441; Example 356, p. 612). In addition, the following preparation processes are disclosed for compound (I) in WO®2014/143799 (Scheme 1)

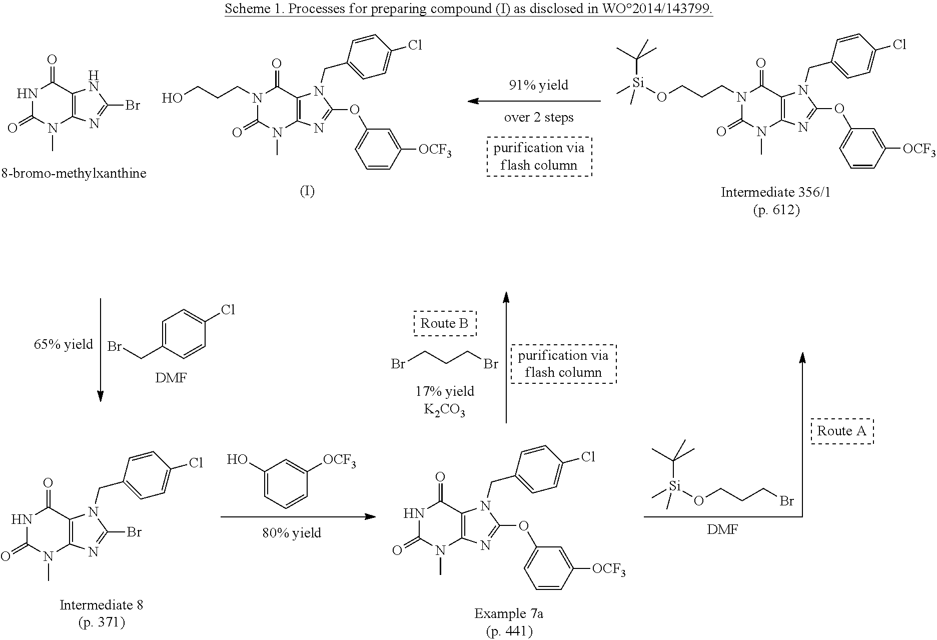

The synthetic approaches as disclosed in WO^{∘}2014/143799 are however not suitable for a large-scale manufacturing process because of the following issues:
1) low yields,
2) involvement of protecting group chemistry (expensive reagents and additional process steps increasing manufacturing costs),
3) use of toxic solvents and reagents (safety issues as well as risks of residual impurities in the drug substance (I)),
4) requirement of liquid chromatography to isolate compound (I).

Therefore, the present invention has the objective to provide an efficient process for preparing compound (I) that is suitable for an industrial scale operation.

### DETAILED DESCRIPTION OF THE INVENTION

The present invention provides the following process for preparing compound (I) (Scheme 2).

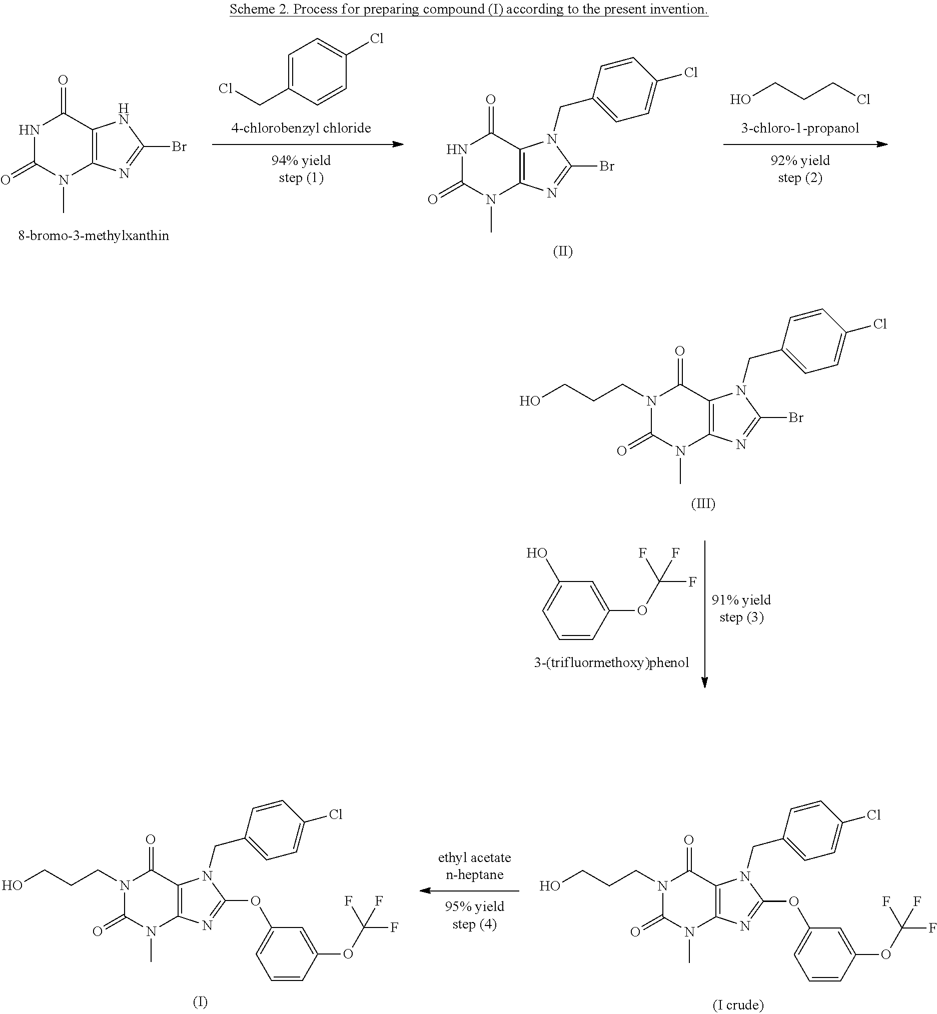

The process according to the present invention makes use of commercially available bulk chemicals that are easy to handle safely.

In one embodiment, the present invention relates to a process for the preparation of compound (I)

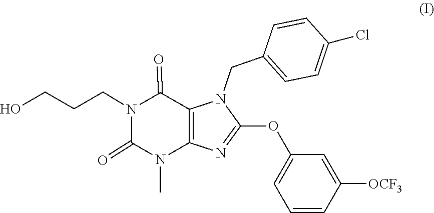 [SMILES: Cn1c(=O)n(CCCO)c(=O)c2c1nc(Oc1cccc(OC(F)(F)F)c1)n2Cc1ccc(Cl)cc1]

comprising following steps:
(1) reacting 8-bromo-3-methylxanthine

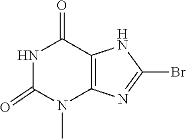

in the presence of a suitable base, in a suitable solvent, with 4-chlorobenzyl chloride to yield compound (II):

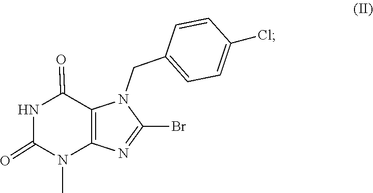 [SMILES: Cn1c(=O)[nH]c(=O)c2c1nc(Br)n2Cc1ccc(Cl)cc1]

(2) reacting compound (II) in the presence of a suitable base and a suitable additive, in a suitable solvent, with 3-chloro-1-propanol to yield compound (III):

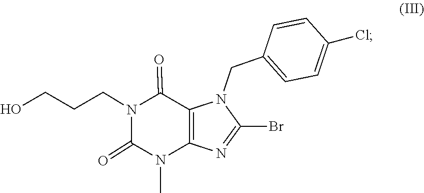 [SMILES: Cn1c(=O)n(CCCO)c(=O)c2c1nc(Br)n2Cc1ccc(Cl)cc1]

and

(3) reacting compound (III) in the presence of a suitable base and a suitable additive, in a suitable solvent, with 3-(trifluoromethoxy)phenole to yield compound (I).

Optionally, the process of the present invention additionally comprises the following step:
(4) recrystallizing compound (I) obtained in step (3) in a suitable solvent.

Thus, in a second embodiment, the present invention relates to a process according to the preceding embodiment further comprising an additional step (4), which is the recrystallization of compound (I) in a suitable solvent.

The process of the present invention involves the isolation and purification of the respective (intermediate) reaction products in crystalline form. That is, compounds (I), (II) and (III) can be isolated from the respective reaction mixture by means of crystallization. In one embodiment, compound (I) can readily be isolated from the reaction mixture by means of crystallization.

Compound (I) is obtained in the process of the present invention with an excellent overall yield of 79% (over 3 steps) and a high purity (98.5%).

Compound (I) is obtained in the process of the present invention after recrystallization with an excellent overall yield of 75% (over 4 steps) and a high purity of 99.8%.

Compound (I) is obtained in the process of the present invention (over 3 steps or over 4 steps) in a form of a stable polymorph, which is identical with the polymorphic form of compound (I) obtainable in the processes as disclosed in WO^{∘}2014/143799.

Where reference is made to the crystallization or the recrystallization of any of the compounds obtained during or by the process of the present invention, the crystallization process may be aided by using an antisolvent or by the addition of seed crystals of the respective compound.

In a preferred embodiment, compound (II) is isolated at the end of reaction step (1) by using an antisolvent, and compounds (III) and (I) are crystallized at the end of reaction steps (2) and (3), respectively, by adding seed crystals. Further preferred, compound (I) is recrystallized in step (4) by adding seed crystals.

Suitable solvents in step (1) include DMAc, DMF, NMP, acetonitrile, acetone, sulfolane, DMSO, chlorinated hydrocarbons such as dichloromethane, cyclic ureas such as DMPU, HMPT, THF; Et₂O, nitromethane, dimethyl- and diethylcarbonate, and ionic liquids; preferably DMAc, DMF, NMP, sulfolane, DMSO, and acetonitrile; most preferably DMAc.

Suitable bases in step (1) include tertiary amines such as triethylamine, diisopropylethylamine, alkylated piperidines, alkylated pyrolidines, inorganic bases such as Na₂CO₃, K₂CO₃, Cs₂CO₃, K₃PO₄, Na₃PO₄, K₂HPO₄, Na₂HPO₄, NaOH, NaOtBu, KOtBu, NaOtAmyl, KOtAmyl or amidines such as DBU and DBN; preferably diisopropylethylamine, Na₂CO₃, K₂CO₃, and K₃PO₄, Na₃PO₄; most preferably diisopropylethylamine.

The reaction temperature in step (1) should be in the range of 50-160° C.; preferably 75-95° C.; most preferably 80-90° C.

The reaction in step (1) is monitored by in-process control analysis. The reaction time in step (1) is typically in the range between 2-12 h; more preferably 3-6 h.

Suitable solvents in step (2) include DMF, DMAc. NMP, acetonitrile, acetone, sulfolane, DMSO, chlorinated hydrocarbons such as dichloromethane, cyclic ureas such as DMPU, HMPT, THF, nitromethane, dimethyl- and diethylcarbonate, and ionic liquids; preferably DMAc, DMF, NMP, sulfolane, DMSO, and acetonitrile; most preferably DMAc.

Suitable bases in step (2) include inorganic bases such as NaHCO₃, KHCO₃, Na₂CO₃, K₂CO₃, Cs₂CO₃, KaPO₄, Na₃PO₄, K₂HPO₄, Na₂HPO₄, NaOH, NaOAc; alcoholates such as NaOtBu, KOtBu, NaOtAmyl, KOtAmyl; and tertiary amines such as diisopropylethylamine, triethylamine, alkylated piperidines, alkylated pyrolidines, and amidines such as DBU and DBN; preferably NaHCO₃, KHCO₃, Na₂CO₃, K₂CO₃, K₃PO₄, Na₃PO₄, K₂HPO₄, Na₂HPO₄, diisopropylethylamine; most preferably NaHCO₃.

Suitable additives in step (2) include quaternary ammonium salts such as tetra-n-butylammonium chloride, tetra-n-butylammoniumbromide, benzyltriethylammonium bromide or chloride, methyltricaprylammonium bromide or chloride, methyltributylammonium bromide or chloride, methyltrioctylammonium bromide or chloride, and organic phosphonium salts such as hexadecyltributylphosphonium bromide or chloride; preferably tetra-n-butylammonium chloride, tetra-n-butylammoniumbromide, benzyltriethylammonium bromide or chloride, methyltributylammonium bromide or chloride; most preferably tetra-n-butylammoniumbromide.

The reaction temperature in step (2) should be in the range of 80-160° C., preferably 100-120° C., most preferably 105-115° C.

The reaction in step (2) is monitored by in-process control analysis. The reaction time in step (2) is typically in the range of 2-12 h, more preferably 4-6 h.

The reaction pressure in step (2) should be kept at 100-1000 mbar (abs.), preferably at 200-400 mbar (abs.), most preferably 250-350 mbar (abs.).

Suitable solvents in step (3) include DMAc, DMF. NMP, acetonitrile, acetone, sulfolane, DMSO, chlorinated hydrocarbons such as dichloromethane, cyclic urea-based solvents such as DMPU, HMPT, THF, Et₂O, nitromethane, dimethyl- and diethylcarbonate, and ionic liquids; preferably DMAc, DMF, NMP, sulfolane, DMSO, and acetonitrile; most preferably NMP.

Suitable bases in step (3) include inorganic bases such as Na₂CO₃, K₂CO₃, Cs₂CO₃, K₃PO₄, Na₃PO₄, K₂HPO₄, Na₂HPO₄, NaOH, NaOAc, NaOtBu, KOtBu, NaOtAmyl, KOtAmyl, tertiary amines such as diisopropylethylamine, triethylamine, alkylated piperidines, alkylated pyrolidines, and amidines such as DBU and DBN; preferably diisopropylethylamine, Na₂CO₃, K₂CO₃, K₃PO₄ and Na₃PO₄; most preferably Na₂CO₃.

Suitable additives in step (3) include quaternary ammonium salts such as tetra-n-butylammonium chloride, tetra-n-butylammoniumbromide, benzyltriethylammonium bromide or chloride, methyltricaprylammonium bromide or chloride, methyltributylammonium bromide or chloride, methyltrioctylammonium bromide or chloride and organic phosphonium salts, such as hexadecyltributylphosphonium bromide or chloride; preferably tetra-n-butylammonium chloride, tetra-n-butylammoniumbromide, benzyltriethylammonium bromide or chloride, methyltributylammonium bromide or chloride; most preferably tetra-n-butylammoniumbromide.

The reaction temperature in step (3) should be in the range of 80-160° C.; preferably 100-130° C.; most preferably 115-125° C.

The reaction time in step (3) is typically in the range of 4-12 h; more preferably 8-10 h.

The reaction pressure in step (2) should be kept at 100-1000 mbar (abs.); preferably at 200-400 mbar (abs.); most preferably 250-350 mbar (abs.).

An additional embodiment of the invention is directed to a process for the preparation of compound (I)

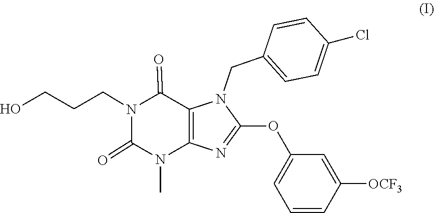 [SMILES: Cn1c(=O)n(CCCO)c(=O)c2c1nc(Oc1cccc(OC(F)(F)F)c1)n2Cc1ccc(Cl)cc1]

comprising the step of reacting compound (III)

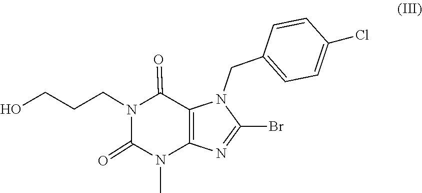 [SMILES: Cn1c(=O)n(CCCO)c(=O)c2c1nc(Br)n2Cc1ccc(Cl)cc1]

in the presence of a suitable base and a suitable additive, in a suitable solvent, with 3-(trifluoromethoxy)phenole to yield compound (I), and optionally further comprising recrystallizing the obtained compound (I) in a suitable solvent.

An additional embodiment of the invention is directed to a process for the preparation of a compound (III)

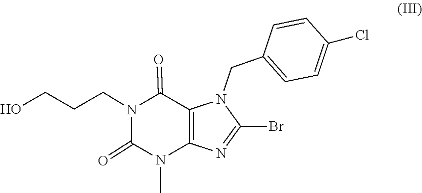 [SMILES: Cn1c(=O)n(CCCO)c(=O)c2c1nc(Br)n2Cc1ccc(Cl)cc1]

comprising the step of reacting compound (II)

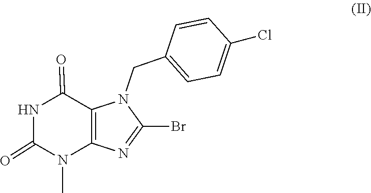 [SMILES: Cn1c(=O)[nH]c(=O)c2c1nc(Br)n2Cc1ccc(Cl)cc1]

in the presence of a suitable base and a suitable additive, in a suitable solvent, with 3-chloro-1-propanol to yield compound (III):

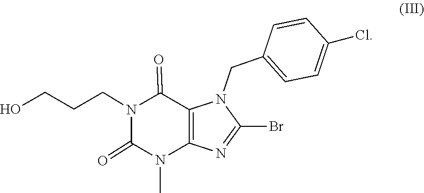 [SMILES: Cn1c(=O)n(CCCO)c(=O)c2c1nc(Br)n2Cc1ccc(Cl)cc1]

The process according to the present invention is superior to the processes as described in WO2014/143799 in that
1) it yields compound (I) in high purity without the need of chromatographic separation and purification steps with a significantly higher yield of 79% (or 75% after recrystallization) vs. 46% yield (route A) or 9% yield (route B) [Table. 1],
2) it does not require protecting group chemistry operations,
3) it limits the use toxic solvents and reagents.

TABLE 1
Yield and purity of compound (I) obtained in the process
as described inWO2014/143799 versus present invention

| | Process as described in | | Process according |
|---|---|---|---|
| | WO°2014/143799 | | to the present |
| Parameter | Route A | Route B | invention |
| Total Yield | 46% | 9% | 79% |
| over all Steps | | | 75% (after |
| | | | recrystallization) |
| Purification | column | HPLC | Crystallization |
| Method | chromatography | chromatography | |
| Purity of | n.a. | n.a. | 98.5% |
| compound (I) | | | 99.8% (after |
| | | | crystallization) |

In a further embodiment, the present invention thus relates to a process for preparing compound (I) as defined in the foregoing, wherein compound (I) is obtained with a purity of 98.5% or more, preferably 99.8% or more.

In yet another embodiment, the present invention relates to compound (I) obtained by the process described herein and characterized by a purity of 98.5% or more, preferably 99.8% or more.

The process according to the present invention is suitable for scale-up to a routine industrial scale production of compound (I) with a batch size of 150 kg and above. Data in Table 2 demonstrates that the process according to the present invention is robust and up-scalable without compromising yields and drug substance quality.

TABLE 2
Comparison of Yields and Purities Lab-Scale to Production Scale

| | | Production scale batches |
|---|---|---|
| | | (Average over all batches, |
| | | scale-up factor of |
| Step | Lab-Scale | 5,000-10,000) |
| Step (1)/Compound (II) | 94% yield | 93% yield |
| | 99.9% purity | 99.9% purity |
| Step (2)/Compound (III) | 92% yield | 93% yield |
| | 98.7% purity | 99.3% purity |
| Step (3)/Compound (I) | 91% yield | 86% yield |
| | 98.5% purity | 99.2% purity |
| Step (4)/Compound (I) | 95% yield | 94% yield |
| | 99.8% purity | 99.9% purity |

### EXPERIMENTAL SECTION

### List of Abbreviations

APCI Atmospheric Pressure Chemical Ionization
abs. absolute
aq. aqueous
BHT 3,5-di-tert-butyl-4-hydroxytoluol
conc concentrated
DCM dichloromethane
DIPEA N-ethyl-diisopropylamine
DMAC dimethylacetamide
DMF dimethylformamide
DMSO dimethylsulfoxide
equiv. equivalents
ESI Electrospray Ionization
EtOAc ethyl acetate
g gram
h hour(s)
HOAc acetic acid
HPLC high performance liquid chromatography
iPr iso-propyl
kg kilogram
NMP N-methyl-2-pyrrolidon
NMR Nuclear Magnetic Resonance
MeCN acetonitrile
MeOH methanol
min minute(s)
mg milligram
mL milliliter
M Molar (mol/L)
TBABr tetra-n-butylammonium bromide
THF tetrahydrofuran

NMR method: NMR spectra were recorded on a Bruker AVANCE III instrument with a frequence of 600 MHz for ¹H-NMR experiments, respectively 150 MHz for 13C-NMR experiments and using TopSpin 3.2 pl6 software for analysis. Chemical shifts are given in parts per million (ppm) downfield from internal reference trimethylsilane in 0 units. Selected data are reported in the following manner: chemical shift (multiplicity, coupling constants (J), number of hydrogens). Abbreviations are as follows: s (singulet), d (doublet), t (triplet), q (quartet), spt (septet), m (multiplet), br (broad).

### X-Ray Powder Diffraction (XRPD) Diagram:

X-ray powder diagrams were generated using a Bruker D8 Advance-diffractometer in reflection mode fitted with a LynxEye Position Sensitive detector—and a Cu-anode as X-ray source with CuKα1 radiation (λ=1.54060 Å, 40 KV, 40 mA). The standard error range for the 2-theta values is +0.2°.

### Examples

### Preparation of Compound (I)

### Step (1): Preparation of 8-bromo-7-(4-chlorobenzyl)-3-methyl-3,7-dihydro-1H-purine-2,6-dione (II)

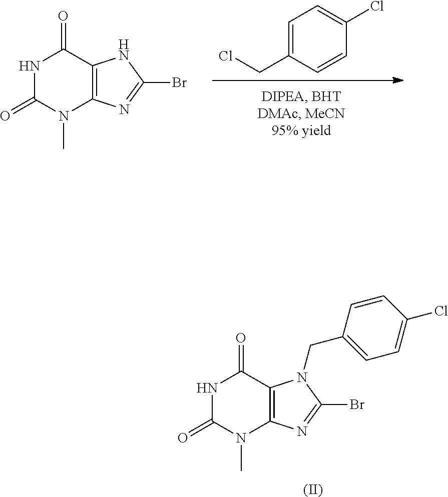 [SMILES: Cn1c(=O)[nH]c(=O)c2[nH]c(Br)nc21 ; ClCc1ccc(Cl)cc1 ; Cn1c(=O)[nH]c(=O)c2c1nc(Br)n2Cc1ccc(Cl)cc1 ; "(II)"]

8-Bromo-3-methylxanthine (20.0 g, 81.6 mmol, 1.0 equiv.) and BHT (0.8 g, 3.6 mmol, 0.04 equiv.) are dissolved in dimethylacetamide (210 mL). The mixture is heated up to 85° C. A solution of 4-chlorobenzyl chloride (15.8 g, 97.9 mmol, 1.2 equiv.) in dimethylacetamide (20 mL) is added and rinsed with dimethylacetamide (10 mL). Diisopropylethylamine (11.1 g, 85.7 mmol, 1.05 equiv.) is added and rinsed with dimethylacetamide (10 mL). The reaction is stirred at 85° C. until the starting material is consumed (8-bromo-3-methylxanthine <0.3%). Optionally, further dosage of diisopropylethylamine (0.5 g, 4.1 mmol, 0.05 equiv.) may be performed to complete the reaction. After complete conversion, hydrochloric acid (4M, 0.8 g. 8.2 mmol, 0.1 eq) is added. The reaction solvent is removed partially via vacuum distillation (until a remaining of approx. 150 mL reaction mixture volume). Acetonitrile (150 mL) is added, and the product suspension is slowly cooled to 20° C. The product is isolated via filtration and the filter cake is washed two times with acetonitrile (50 mL). The isolated material is dried under reduced pressure at 50° C. giving compound (II) (28.7 g, 78.0 mmol, 95% yield, 99.9% purity) as a colorless solid. Melting point: 270-271 ºC.

### Analytical Data

¹H NMR (DMSO-d₆) δ: 11.37 (s, 1H), 7.44 (d, J=8.5 Hz, 2H). 7.29 (d, J=8.5 Hz, 2H), 5.48 (s, 2H), 3.34 (s, 3H): ¹³C NMR (DMSO-d₆) δ: 154.0, 150.5, 149.3, 134.5, 132.6, 129.0, 128.7, 127.9, 108.6, 48.6, 28.5; HRMS (ESI): m/z 369, ([M+H]⁺, exp. 368.9763, calc. 368.9748.

### Step (2): Preparation of 8-bromo-7-(4-chlorobenzyl)-1-(3-hydroxypropyl)-3-methyl-3,7-dihydro-1H-purine-2,6-dione (III)

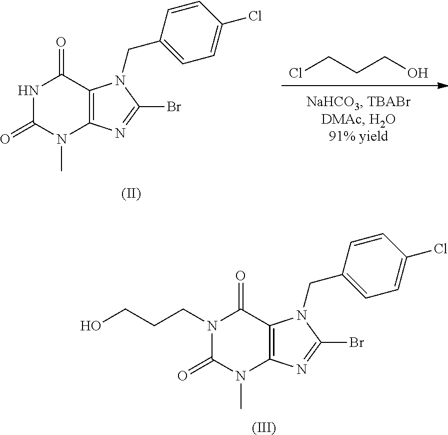

Compound (II) (20.0 g, 54.1 mmol, 1.0 equiv.) and sodium bicarbonate (6.8 g, 81.2 mmol, 1.5 equiv.), tetrabutylammonium bromide (0.8 g, 2.7 mmol, 0.05 equiv.) are suspended in dimethylacetamide (170 mL). The mixture is heated to 110° C. 3-chloro-1-propanol (7.7 g, 81.5 mmol, 1.5 equiv.) is added, and rinsed with dimethylacetamide (10 mL). Vacuum (200-400 mbar) is then applied. The reaction is stirred at 110° C. until the starting material is consumed (compound (II)<0.5%). After complete conversion, the reaction mixture is cooled to 80° C., filtered and rinsed with dimethylacetamide (30 mL). Water (160 mL) is added to the filtrate at 90° C., then sodium bicarbonate (0.5 g, 5.4 mmol, 0.1 equiv.) is added. The mixture is cooled to 70° C. and seed crystals (47 mg) are added. The crystal suspension is cooled to 40° ° C. over 60 min, heated to 70° C., kept for at least 15 min at 70° C. and cooled to 20° C. over 150 min and stirred for 1 h. The product is isolated via filtration and the filter cake is washed with water (160 mL). The isolated material is dried under reduced pressure at 60° ° C. giving compound (III) (16.1 g, 37.7 mmol, 91% yield, 98.7% purity) as a colorless solid. Melting point: 148-149° C.

### Analytical Data

¹H NMR (DMSO-d₆) δ: 7.43 (d, J=8.5 Hz, 2H), 7.30 (d, J=8.5 Hz, 2H), 5.52 (s, 2H), 4.48 (t, J=5.2 Hz, 1H), 3.89-3.96 (m, 2H), 3.43-3.48 (m, 2H), 3.38 (s, 2H), 1.65-1.74 (m, 2H): ¹³C NMR (DMSO-d₆) δ: 153.5, 150.3, 147.9, 134.5, 132.6, 129.0, 128.7, 128.2, 108.2, 58.7, 48.7, 38.5, 30.8, 29.5; HRMS (ESI): m/z 427, ([M+H]⁺, exp. 427.0188, calc. 427.0167.

### Step (3): Preparation of 7-(4-chlorobenzyl)-1-(3-hydroxypropyl)-3-methyl-8-(3-(trifluoromethoxy)phenoxy)-3,7-dihydro-1H-purine-2,6-dione (I) Crude]

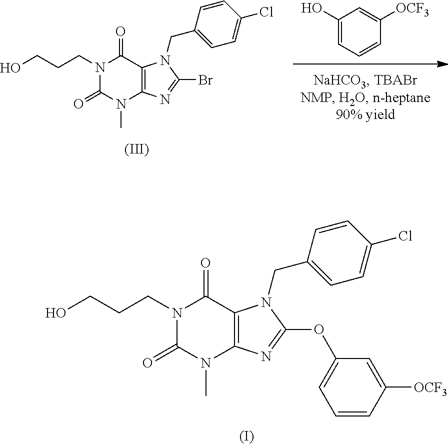

Compound (III) (20.0 g, 46.8 mmol, 1.0 equiv.), tetrabutylammonium bromide (0.8 g. 2.4 mmol, 0.05 equiv.) and sodium carbonate (3.5 g, 32.7 mmol, 0.7 equiv.) are suspended in N-methyl-2-pyrrolidone (135 mL). The mixture is heated to 50° C. 3-(Trifluoromethoxy)phenole (9.2 g, 51.7 mmol, 1.1 eq) is added and rinsed with N-methyl-2-pyrrolidone (10 mL). The mixture is heated to 120° C. and stirred at 120° C. under reduced pressure (200-400 mbar) until the starting material is consumed (compound (III)<1.0%). After complete conversion, the reaction mixture is cooled to 80° C., filtered and rinsed with N-methyl-2-pyrrolidone (15 mL). Acetonitrile (40 mL) is added. Water (110 mL) is added in at least 30 min The mixture is cooled to 58° C. and seed crystals (20 mg) are added. The crystal suspension is consecutively cooled to 40° C., heated to 60° C., kept for at least 15 min at same temperature and cooled to 20° C. The product is isolated via filtration and the filter cake is washed with water (160 mL) and n-heptane (40 mL). The isolated material is dried under reduced pressure at 60° ° C. giving compound (I) crude (22.1 g, 42.1 mmol, 90% yield, 98.5% purity) as a colorless solid. Melting point: 124-125° C.

### Analytical Data

¹H NMR (DMSO-d₆) δ: 7.56-7.63 (m, 1H), 7.49 (s, 1H), 7.40-7.45 (m, 5H), 7.32 (br d, J=8.3 Hz, 1H), 5.44 (s, 2H), 4.47 (t, J=5.3 Hz, 1H), 3.87-3.96 (m, 2H), 3.39-3.48 (m, 2H), 3.29 (s, 3H), 1.63-1.75 (m, 2H); ¹³C NMR (DMSO-d₆) δ: 153.8, 153.7, 152.1, 150.5, 148.6, 145.4, 135.1, 132.5, 131.3, 129.5, 128.7, 118.8, 118.2, 119.9, 113.1, 102.5, 58.7, 45.8, 38.3, 30.9, 29.5; HRMS (ESI): m/z 525 ([M+H]⁺, exp. 525.1151, calc. 525.1147).

### Step (4): Recrystallization of 7-(4-chlorobenzyl)-1-(3-hydroxypropyl)-3-methyl-8-(3-(trifluoromethoxy)phenoxy)-3,7-dihydro-1H-purine-2,6-dione (I)

Compound (I) (20.0 g, 38.1 mmol, 1.0 equiv.) is suspended in ethyl acetate (90 mL). The mixture is heated to 65° C., filtered, and n-heptane (100 mL) is added to the solution. The mixture is cooled to 53° C. and seed crystals (40 mg) are introduced. The crystalline suspension is stirred at least 60 min before n-heptane (100 mL) is being added and stirred another 60 min at 53° C. The suspension is cooled to 5° ° C. over 90 min, and stirred 120 min at 5° C. The product is isolated via filtration and the filter cake is washed with n-heptane (100 mL). The isolated material is dried under reduced pressure at 50° ° C. giving compound (I) (18.9 g, 36.2 mmol, 95% yield, 99.8% purity) as a colorless solid. Melting point: 124° C.

### Analytical Data

¹H NMR (DMSO-d₆) δ: 7.56-7.63 (m, 1H), 7.49 (s, 1H), 7.40-7.45 (m, 5H), 7.32 (br d, J=8.3 Hz, 1H), 5.44 (s, 2H), 4.47 (t, J=5.3 Hz, 1H), 3.87-3.96 (m, 2H), 3.39-3.48 (m, 2H), 3.29 (s, 3H), 1.63-1.75 (m, 2H); ¹³C NMR (DMSO-d₆) δ: 153.8, 153.7, 152.1, 150.5, 148.6, 145.4, 135.1, 132.5, 131.3, 129.5, 128.7, 118.8, 118.2, 119.9, 113.1, 102.5, 58.7, 45.8, 38.3, 30.9, 29.5; HRMS (ESI): m/z 525 ([M+H]⁺, exp. 525.1150, calc. 525.1147).

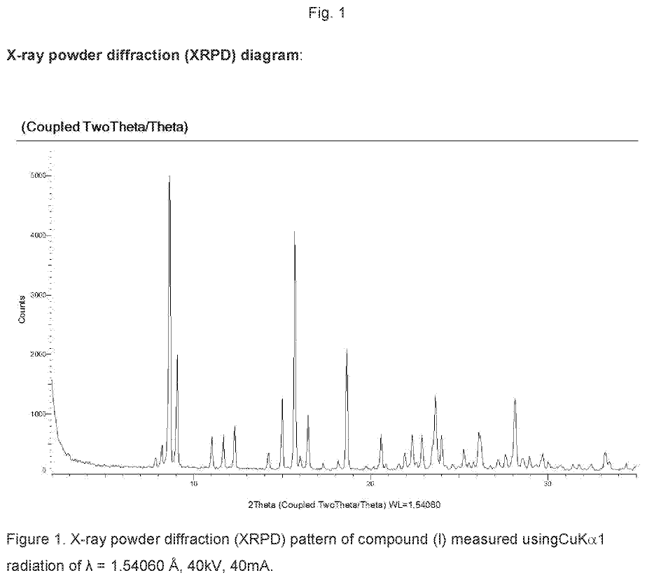

The recrystallized compound (I) prepared according to Step (4) was analyzed by X-ray powder diffraction to obtain the results as shown in FIG. 1 (XRPD-diagram) and Table 1 set forth below.

TABLE I
XRPD pattern peak data for compound (I).

| No. | 2Theta, ° | Rel. Intensity |
|---|---|---|
| 1 | 8.7 ± 0.2 | 100 |
| 2 | 9.1 ± 0.2 | 40.8 |
| 3 | 11.7 ± 0.2 | 12.5 |
| 4 | 12.3 ± 0.2 | 13.5 |
| 5 | 15.0 ± 0.2 | 26.3 |
| 6 | 15.7 ± 0.2 | 84 |
| 7 | 16.5 ± 0.2 | 19 |
| 8 | 18.7 ± 0.2 | 43.7 |
| 9 | 23.7 ± 0.2 | 19 |
| 10 | 28.1 ± 0.2 | 25.4 |

### Preparation of 3-(trifluoromethoxy)phenol

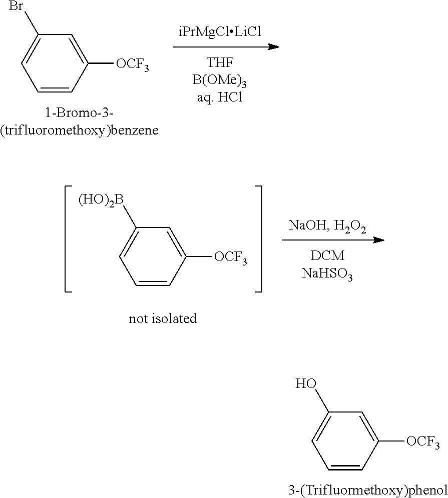

1-Bromo-3-(trifluoromethoxy)benzene (70.0 g, 290 mmol, 1.0 equiv.) was dissolved in THF (17 mL) at room temperature and iPrMgBr·LiCl (14 wt.-% in THF, 298 g, 405 mmol, 1.4 equiv.) was added within 20 min at 15° C. After complete addition, the reaction mixture was stirred 2.5 h at room temperature until complete conversion (1-Bromo-3-(trifluoromethoxy)benzene <3.0%). The reaction mixture was then cooled to 0° C. and consecutively quenched first by addition of B(OMe)₃ (31.8 g, 304 mmol, 1.1 equiv.), addition of water (105 mL), followed by aqueous HCl (4M, 45.4 g, 461 mmol, 1.6 equiv.). After phase separation, the reaction solvent of the organic phase is removed partially via vacuum distillation (to approx. 80 mL remaining reaction mixture volume). Water (240 mL) and sodium hydroxide solution (45 wt.-%, 39.6 g, 446 mmol, 1.5 equiv.) is added to the mixture, and the reaction mixture is further concentrated at 80° C. jacket temperature under reduced pressure (150 mbar) to approx. 260 mL remaining reaction mixture volume. After addition of water (67 mL) and cooling to 5° C., hydrogen peroxide (35 wt.-% in water, 29.6 g, 304 mmol, 1.1 equiv.) was added below 30° C. Upon complete addition, the reaction was stirred upon complete conversion (boronic acid intermediate <0.1%) at 20° C. and then quenched by addition of aqueous sodium sulfite (26.7 g, 29.0 mmol, 0.1 equiv.). After addition of aqueous HCl (4M, 79.2 g, 782 mmol, 2.7 equiv.) and dichloromethane (200 mL), the organic layer was separated. The crude mixture was distilled using a vapour duct and under reduced pressure (72° C. b.p. at 80 mbar) giving 3-(trifluoromethoxy)phenol (46.9 g, 263 mmol, 91% yield) as a colorless liquid.

### Analytical Data

¹H NMR (DMSO-d₆) δ: 10.0 (s, 1H), 7.28 (m, 1H), 6.71-6.80 (m, 3H): ¹³C NMR (DMSO-d₆) δ: 158.8, 149.2, 130.7, 120.4, 114.4, 110.9, 107.8; HRMS (neg. APCI): m/z 285 ([M+Cl]⁻, exp. 285.0515, calc. 241.0511.

Alternatively, the intermediate boronic acid is isolated before it is further reacted to yield 3-(trifluoromethoxy)phenol in a process comprising two consecutive steps:

### Preparation of (3-(trifluoromethoxy)phenyl)boronic Acid

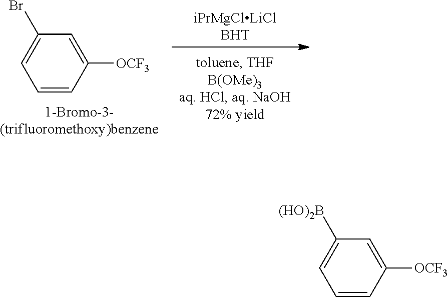

1-Bromo-3-(trifluoromethoxy)benzene (25.0 g, 103 mmol, 1.0 equiv.), BHT (0.6 g, 3 mmol, 0.02 equiv.) was dissolved in THF (6 mL) at room temperature and iPrMgBr LiCl (14 wt.-% in THF, 114 g, 155 mmol, 1.5 equiv.) was added within 15 min at 15° C. After complete addition, the reaction mixture was stirred 2 h at 30° C. until complete conversion (1-Bromo-3-(trifluoromethoxy)benzene <3.0%). The reaction mixture was then cooled to 0° C. and quenched by addition of B(OMe)₃ (11.5 g. 109 mmol, 1.05 equiv.) until complete conversion ((trifluoromethoxy)benzene <2.0%) and diluted with water (38 mL) and toluene (25 mL). The reaction mixture was then quenched by adding to aqueous HCl (4M, 16.2 g, 164 mmol, 1.6 equiv.) at 0° C., whereafter phases were separated. The organic layer was again washed with aqueous HCl (1M, 40 mL) and the resulting organic layer was diluted with toluene (78 mL) and concentrated (THF <5.0%). After phase separation, the organic layer was treated consecutively with aqueous sodium hydroxide solution (6 wt.-%, 73.4 g, 68 mL, 144 mmol, 1.1 equiv.) and after phase separation the organic layer was washed again with aqueous sodium hydroxide solution (6 wt.-%, 26.7 g, 41 mL, 41 mmol, 0.4 equiv.). The combined aqueous phases were acidified with aqueous HCl (4M, 16.3 g, 165 mmol, 1.6 equiv.) at room temperature to a pH of 1-3 and cooled to 0° C. The desired product was obtained by filtration, and the filter cake was washed with water (40 mL), dried and the desired title compound was obtained as colorless crystals (15.3 g, 74 mmol, 72% yield). Melting point: 86° C.

### Analytical Data

¹H NMR (DMSO-d⁶) δ: 10.05 (br s, 1H), 7.29 (t, J=8.2 Hz, 1H), 6.82 (ddd, J=8.2, 2.3, 0.8 Hz, 1H), 6.74-6.76 (m, 1H), 6.72-6.74 (m, 1H); ¹³C NMR (DMSO-d⁶) δ: 159.3, 149.7, 131.1, 120.6 (q, J=121 Hz), 114.9, 111.3, 108.3; HRMS (ESI): m/z 241 ([M+Cl]⁻, exp. 241.0058, calc. 241.0056.

### Preparation of 3-(trifluoromethoxy)phenol from (3-(trifluoromethoxy)phenyl)boronic Acid

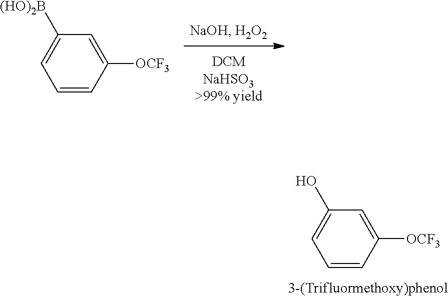

(3-(trifluoromethoxy)phenyl)boronic acid (69.2 g, 336 mmol, 1.0 equiv.) was dissolved in water (250 ml) and treated with aqueous sodium hydroxide solution (50 wt.-%, 37.7 g, 471 mmol, 1.4 equiv.) at room temperature. Hydrogen peroxide (30 wt.-% in water, 43.9 g, 38 mL, 387 mmol, 1.2 equiv.) was added below 30° C. and dissolved with water (40 mL) upon complete addition. The reaction was stirred upon complete conversion (boronic acid intermediate <0.1%) at 15° C. and then quenched by consecutively addition of aqueous sodium sulfite (152 g, 165 mmol, 0.5 equiv.) and aqueous HCl (4M, 92.0 g, 908 mmol, 2.7 equiv.). The mixture was diluted with dichloromethane (300 mL), and the organic layer was separated. The crude mixture was distilled using a vapour duct and under reduced pressure (72° C. b.p. at 80 mbar) giving 3-(trifluoromethoxy)phenol (59.3 g, 336 mmol, >99% yield) as a colorless liquid.

### Analytical Data

¹H NMR (DMSO-d₆) δ: 10.0 (s, 1H), 7.28 (m, 1H), 6.71-6.80 (m, 3H); ¹³C NMR (DMSO-d₆) δ: 158.8, 149.2, 130.7, 120.4, 114.4, 110.9, 107.8; HRMS (neg. APCI): m/z 285 ([M+Cl]⁻, exp. 285.0515, calc. 241.0511.

1. A process for preparing compound (I) 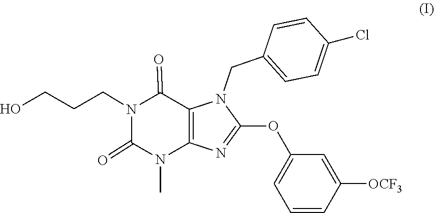 [SMILES: Cn1c(=O)n(CCCO)c(=O)c2c1nc(Oc1cccc(OC(F)(F)F)c1)n2Cc1ccc(Cl)cc1]
comprising following steps:
(1) reacting 8-bromo-3-methylxanthine 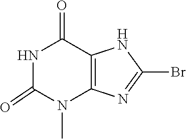
in the presence of a suitable base, in a suitable solvent, with 4-chlorobenzyl chloride;
to yield compound (II) 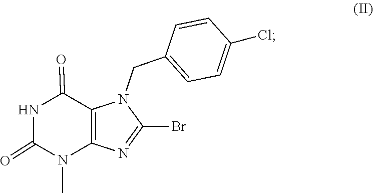 [SMILES: Cn1c(=O)[nH]c(=O)c2c1nc(Br)n2Cc1ccc(Cl)cc1]
(2) reacting compound (II) in the presence of a suitable base and a suitable additive, in a suitable solvent, with 3-chloro-1-propanol,
to yield compound (III) 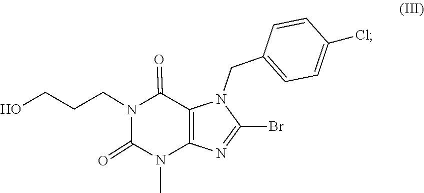 [SMILES: Cn1c(=O)n(CCCO)c(=O)c2c1nc(Br)n2Cc1ccc(Cl)cc1]
reacting compound (III) in the presence of a suitable base and a suitable additive, in a suitable solvent, with 3-(trifluoromethoxy)phenole
to yield compound (I).

2. The process according to claim 1, wherein compound (I) is isolated from the reaction mixture by means of crystallization.

3. The process according to claim 1, further comprising a step (4) comprising recrystallizing compound (I) in a suitable solvent.

4. The process according to claim 1, wherein the solvent in step (1) is chosen from the group consisting of DMAc, DMF, NMP, acetonitrile, acetone, sulfolane, DMSO, chlorinated hydrocarbons such as dichloromethane, cyclic ureas such as DMPU, HMPT, THF, Et₂O, nitromethane, dimethyl- and diethylcarbonate, and ionic liquids; preferably DMAc, DMF, NMP, sulfolane, DMSO, and acetonitrile; most preferably DMAc.

5. The process according to claim 1, wherein the base in step (1) is chosen from the group consisting of tertiary amines such as triethylamine, diisopropylethylamine, alkylated piperidines, alkylated pyrolidines, inorganic bases such as Na₂CO₃, K₂CO₃, Cs₂CO₃, K₃PO₄, Na₃PO₄, K₂HPO₄, Na₂HPO₄, NaOH, NaOtBu, KOtBu, NaOtAmyl, KOtAmyl or amidines such as DBU and DBN; preferably diisopropylethylamine, Na₂CO₃, K₂CO₃, and K₃PO₄, Na₃PO₄; most preferably diisopropylethylamine.

6. The process according to claim 1, wherein the reaction temperature in step (1) is in the range of 75-95° C.; most preferably 80-90° C.

7. The process according to claim 1, wherein the solvent in step (2) is selected from the group consisting of DMF, DMAc, NMP, acetonitrile, acetone, sulfolane, DMSO, chlorinated hydrocarbons such as dichloromethane, cyclic ureas such as DMPU, HMPT, THF, nitromethane, dimethyl- and diethylcarbonate, and ionic liquids; preferably DMAc, DMF, NMP, sulfolane, DMSO, and acetonitrile; most preferably DMAc.

8. The process according to claim 1, wherein the base in step (2) is selected from the group consisting of inorganic bases such as NaHCO₃, KHCO₃, Na₂CO₃, K₂CO₃, Cs₂CO₃, K₃PO₄, Na₃PO₄, K₂HPO₄, Na₂HPO₄, NaOH, NaOAc;
alcoholates such as NaOtBu, KOtBu, NaOtAmyl, KOtAmyl; and tertiary amines such as diisopropylethylamine, triethylamine, alkylated piperidines, alkylated pyrolidines, and amidines such as DBU and DBN; preferably NaHCO₃, KHCO₃, Na₂CO₃, K₂CO₃, K₃PO₄, Na₃PO₄, K₂HPO₄, Na₂HPO₄, diisopropylethylamine; most preferably NaHCO₃.

9. The process according to claim 1, wherein the additive in step (2) is selected from the group consisting of quaternary ammonium salts such as tetra-n-butylammonium chloride, tetra-n-butylammoniumbromide, benzyltriethylammonium bromide or chloride, methyltricaprylammonium bromide or chloride, methyltributylammonium bromide or chloride, methyltrioctylammonium bromide or chloride, and organic phosphonium salts such as hexadecyltributylphosphonium bromide or chloride; preferably tetra-n-butylammonium chloride, tetra-n-butylammoniumbromide, benzyltriethylammonium bromide or chloride, methyltributylammonium bromide or chloride; most preferably tetra-n-butylammoniumbromide.

10. The process according to claim 1, wherein the reaction temperature in step (2) is in the range of 100-120° C.; most preferably 105-115° C.

11. The process according to claim 1, wherein the solvent in step (3) is selected from the group consisting of DMAc, DMF, NMP, acetonitrile, acetone, sulfolane, DMSO, chlorinated hydrocarbons such as dichloromethane, cyclic urea based solvents such as DMPU, HMPT, THF, Et₂O, nitromethane, dimethyl- and diethylcarbonate, and ionic liquids; preferably DMAc, DMF, NMP, sulfolane, DMSO, and acetonitrile; most preferably NMP.

12. The process according to claim 1, wherein the base in step (3) is selected from the group consisting of inorganic bases such as Na₂CO₃, K₂CO₃, Cs₂CO₃, K₃PO₄, Na₃PO₄, K₂HPO₄, Na₂HPO₄, NaOH, NaOAc, NaOtBu, KOtBu, NaOtAmyl, KOtAmyl, tertiary amines such as diisopropylethylamine, triethylamine, alkylated piperidines, alkylated pyrolidines, and amidines such as DBU and DBN; preferably diisopropylethylamine, Na₂CO₃, K₂CO₃, K₃PO₄ and Na₃PO₄; most preferably Na₂CO₃.

13. The process according to claim 1, wherein the additive in step (3) is selected from the group consisting of quaternary ammonium salts such as tetra-n-butylammonium chloride, tetra-n-butylammoniumbromide, benzyltriethylammonium bromide or chloride, methyltricaprylammonium bromide or chloride, methyltributylammonium bromide or chloride, methyltrioctylammonium bromide or chloride and organic phosphonium salts, such as hexadecyltributylphosphonium bromide or chloride; preferably tetra-n-butylammonium chloride, tetra-n-butylammoniumbromide, benzyltriethylammonium bromide or chloride, methyltributylammonium bromide or chloride; most preferably tetra-n-butylammoniumbromide.

14. The process according to claim 1, wherein the reaction temperature in step (3) is in the range of 100-130° C.; most preferably 115-125° C.

15. The process according to claim 1, wherein the reaction pressure in step (2) and/or step (3) is kept in the range of 200-400 mbar (abs.); most preferably 250-350 mbar (abs.).

16. The process according to claim 1, wherein compound (I) is obtained with a purity of 98.5% or more, preferably 99.8% or more.
